(19) United States
(12) Patent Application Publication
(10) Pub. No.: US 2009/0215744 A1
(43) Pub. Date: Aug. 27, 2009

(54) Solid Formulations

(75) Inventors: Dan B. Brown, Wilmington, DE (US); Daniel Korey, Wilmington, DE (US); Karen B. Main, Wilmington, DE (US); Richard J.R. Creekmore, Wilmington, DE (US)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/093,956

(22) PCT Filed: Nov. 18, 2006
(86) PCT No.: PCT/US06/61071

§ 371 Date: Oct. 3, 2008

Related U.S. Application Data
(60) Provisional application No. 60/737,863, filed on Nov. 18, 2005.

(51) Int. Cl. A61K 31/554

(52) U.S. Cl. 514/211.13

(57) ABSTRACT

The present invention is directed to solid formulations of the pharmaceutical compound 11-piperazin-1-yldibenzo[b,f][1,4]thiazepine as well as preparations, and pharmaceutical uses thereof.

### FIELD OF THE INVENTION

The present invention is directed to solid formulations containing the pharmaceutical compound 11-piperazin-1-yldibenzo[b,f][1,4]thiazepine, as well as pharmaceutical uses thereof.

### BACKGROUND OF THE INVENTION

A goal of antipsychotic drug development has been to develop agents with increased efficacy and safety along with fewer of the side effects commonly associated with the older antipsychotic medications. Quetiapine fumarate is described in U.S. Pat. No. 4,879,288, which is incorporated herein by reference. Quetiapine fumarate is able to treat both the positive (hallucinations, delusions) and negative symptoms (emotional withdrawal, apathy) of psychosis and is associated with fewer neurological and endocrine related side effects compared to older agents. Quetiapine fumarate has also been associated with a reduction in hostility and aggression. Quetiapine fumarate is associated with fewer side effects such as EPS, acute dystonia, acute dyskinesia, as well as tardive dyskinesia. Quetiapine fumarate has also helped to, enhance patient compliance with treatment, ability to function and overall quality of life, while reducing recidivism. P. Weiden et al., Atypical antipsychotic drugs and long-term outcome in schizophrenia, 11 J. Clin. Psychiatry, 53-60, 57 (1996). Because of quetiapine fumarate's enhanced tolerability profile its use is particularly advantageous in the treatment of patients that are hypersensitive to the adverse effects of antipsychotics (such as elderly patients).

Derivatives of 11-(piperazin-1-yl)dibenzo[b,f][1,4]-thiazepines and related compounds including metabolites of quetiapine were prepared and evaluated in E. Warawa et al. Behavioral approach to nondyskinetic dopamine antagonists: identification of Seroquel, 44, J. Med. Chem., 372-389 (2001). Quetiapine metabolism has been reported in C. L. Devane et al. Clin. Pharmacokinet., 40(7), 509-522 (2001) wherein the structure of 11-piperazin-1-yldibenzo[b,f][1,4]thiazepine (see Formula I below) was shown in FIG. 1. This compound was reported by Schmutz et al. in U.S. Pat. No. 3,539,573. This compound has also been used in processes for preparing quetiapine as reported in U.S. Pat. No. 4,879,288. It has now been found that 11-piperazin-1-yldibenzo[b,f][1,4]thiazepine is a circulating metabolite of quetiapine in humans.

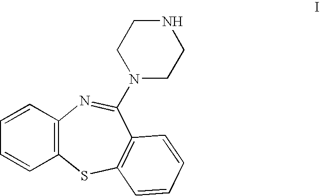

### SUMMARY OF THE INVENTION

The present invention provides solid formulations comprising solid 11-piperazin-1-yldibenzo[b,f][1,4]thiazepine and at least one pharmaceutical excipient selected from a filler, a binder, a disintegrant, a suspending agent, a coating agent, a sweetener, a flavoring, and a lubricant.

The present invention further provides methods of treating at least one symptom or condition associated with but not limited to: 1) Schizophrenia and other Psychotic Disorders including but not limited to Psychotic Disorder, Schizophreniform Disorder, Schizoaffective Disorder, Delusional Disorder, Brief Psychotic Disorder, Shared Psychotic Disorder, and Psychotic Disorder Due to a General Medical Condition; 2) Dementia and other Cognitive Disorders; 3) Anxiety Disorders including but not limited to Panic Disorder Without Agoraphobia, Panic Disorder With Agoraphobia, Agoraphobia Without History of Panic Disorder, Specific Phobia, Social Phobia, Obsessive-Compulsive Disorder, Postraumatic Stress Disorder, Acute Stress Disorder, Generalized Anxiety Disorder and Generalized Anxiety Disorder Due to a General Medical Condition. 4) Mood Disorders including but not limited to a) Depressive Disorders, including but not limited to Major Depressive Disorder and Dysthymic Disorder and b) Bipolar Depression and/or Bipolar mania including but not limited to Bipolar I Disorder, including but not limited to those with manic, depressive or mixed episodes, and Bipolar II Disorder, c) Cyclothymic Disorder, d) Mood Disorder Due to a General Medical Condition; 5) Sleep Disorders; 6) Disorders Usually First Diagnosed in Infancy, Childhood, or Adolescence including but not limited to Mental Retardation, Learning Disorders, Motor Skills Disorder, Communication Disorders, Pervasive Developmental Disorders, Attention-Deficit and Disruptive Behavior Disorders, Feeding and Eating Disorders of Infancy or Early Childhood, Tic Disorders, and Elimination Disorders; 7) Substance-Related Disorders including but not limited to Substance Dependence, Substance Abuse, Substance Intoxication, Substance Withdrawal, Alcohol-Related Disorders, Amphetamine (or Amphetamine-Like)-Related Disorders, Caffeine-Related Disorders, Cannabis-Related Disorders, Cocaine-Related Disorders, Hallucinogen-Related Disorders, Inhalant-Related Disorders, Nicotine-Related Disorders, Opioid-Related Disorders, Phencyclidine (or Phencyclidine-Like)-Related Disorders, and Sedative-, Hypnotic- or Anxiolytic-Related Disorders; 8) Attention-Deficit and Disruptive Behavior Disorders; 9) Eating Disorders; 10) Personality Disorders including but not limited to Obsessive-Compulsive Personality Disorder; and 11) Impulse-Control Disorders, comprising administering to a mammal a therapeutically effective amount of a formulation of the invention.

### BRIEF DESCRIPTION OF THE DRAWINGS

FIG. 1 depicts an XRPD pattern consistent with crystalline Form A.

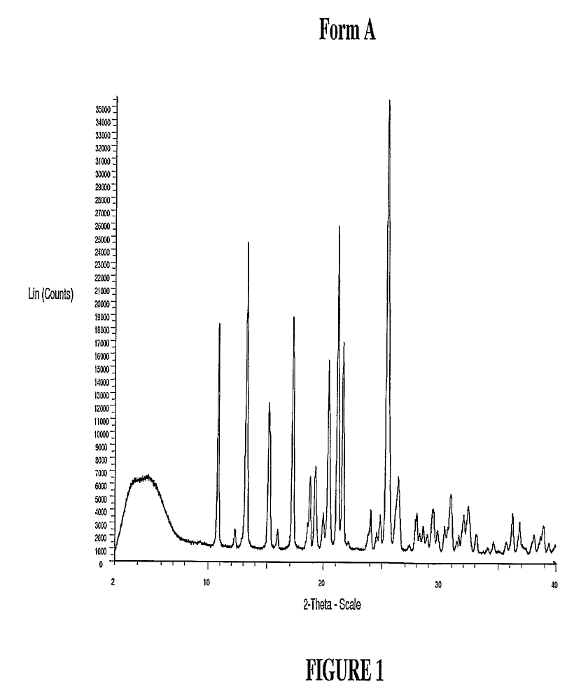

FIG. 2 depicts TGA and DSC data consistent with crystalline Form A.

FIG. 3 depicts DVS data consistent with crystalline Form A.

### DETAILED DESCRIPTION

The compound of Formula I is a dibenzothiazepine that has shown antidopaminergic activity. It has been shown to interact with a broad range of neurotransmitter receptors but has a higher affinity for serotonin (5-HT₂) receptors relative to dopamine (D₂) receptors in the brain. Preliminary positron emission topography (PET) scans of primate subjects showed that the compound of Formula I reaches the brain and occupies D1, D₂, 5-HT_{2A}, and 5-HT_{1A} receptors and the 5HT Transporter. However, the compound of Formula I was not shown to be efficacious in a mouse standard apomorphine swim test (p.o.) and in a rat D-Ampehtamine locomotor activity test (s.c.).

The compound of Formula I has also been shown to have partial 5HT_{1A} agonist activity and has shown in-vivo efficacy in mouse and rat models for depression. The compound of Formula I may be used as an antipsychotic with a reduction in the potential to cause side effects such as acute dystonia, acute dyskinesia, as well as tardive dyskinesia typically seen with antipsychotics. Results generated from alpha receptor binding data further suggest that the compound of Formula I will have improved tolerability over that of quetiapine and suggest that one would observe a reduced incidence of hypotension. Further the compound of Formula I may be used to treat patients of all ages and is advantageous in the treatment of elderly patients.

The present invention provide, inter alia, solid formulations containing the pharmaceutical compound 11-piperazin-1-yldibenzo[b,f][1,4]thiazepine (Formula I). The formulations of the invention can be in solid form, such as in a powder form or a compressed powder suitable, e.g., for oral administration or for the preparation of suspensions. Solid formulations can be prepared by any suitable method including, for example, wet granulation methods.

The solid formulations of the invention can contain 11-piperazin-1-yldibenzo[b,f][1,4]thiazepine in the form of a solid, such as an amorphous solid, crystalline solid, or mixture thereof. In some embodiments, the solid 11-piperazin-1-yldibenzo[b,f][1,4]thiazepine is crystalline having, for example, characteristics of crystalline form A or other crystalline forms.

The solid formulations of the invention can further contain at least one ingredient selected from a filler, a binder, a disintegrant, a suspending agent, a coating agent, a sweetener, a flavoring, a lubricant, or other ingredient. In some embodiments, the solid formulation comprises an excipient selected from an inorganic salt filler, a cellulose filler, an oligosaccharide filler, a non-cellulosic binder, a disintegrant, and a lubricant.

Suitable fillers include, for example, oligosaccharides (e.g., lactose), sugars, starches, modified starches, sugar alcohols (e.g. mannitol, sorbitol, xylitol, lactitol), inorganic salts, cellulose derivatives (e.g. microcrystalline cellulose, silicified microcrystalline cellulose, cellulose, hypromellose), calcium sulfate, aluminum and magnesium silicate complexes and oxides, and the like. An example of an inorganic salt filler is a phosphate salt such as dibasic calcium phosphate dihydrate or salts of sulfates.

Suitable binders include, for example, povidone, lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatin, sodium alginate, and the like. Non-cellulosic binders include polymeric and other binders lacking a cellulose backbone. Examples of non-cellulosic binders include povidone, lactose, starches, modified starches, gums, guar gum, pectin, waxes, gelatins, alginates, and the like.

Suitable disintegrants include, for example, croscarmellose sodium, crospovidone, polyvinylpyrrolidone, sodium starch glycolate, corn starch, microcrystalline cellulose, hydroxypropyl methylcellulose, hydroxypropyl cellulose, and the like.

Suitable lubricants include, for example, magnesium stearate, stearic acid, palmitic acid, calcium stearate, talc, carnauba wax, hydrogenated vegetable oils, mineral oil, polyethylene glycols, sodium stearyl fumarate, and the like.

Sweeteners that may be used include artificial and natural sweeteners such as aspartame, acesulfame potassium, saccharin, saccharin sodium, sucralose, as well as sugar sweeteners such as xylose, ribose, glucose, mannose, galactose, fructose, dextrose, sucrose, maltose, partially hydrolyzed starch (such as maltitol syrup) or corn syrup solids and sugar alcohols such as sorbitol, xylitol, mannitol, glycerin and combinations thereof. Preferably, the type of glycerin used is U.S.P. grade. Preferred as a sugar sweetener is high fructose corn syrup and mixtures thereof.

Flavoring agents that are suitable include, and are not limited to, natural flavors, natural fruit flavors, artificial flavors, artificial fruit flavors, flavor enhancers or mixtures thereof. Natural flavors, artificial flavors or mixtures thereof include, and are not limited to, mint (such as peppermint or spearmint), menthol, cinnamon, vanilla, artificial vanilla, chocolate, artificial chocolate or bubblegum. Natural fruit flavors, artificial fruit flavors or mixtures thereof include, and are not limited to, cherry, grape, orange, strawberry or lemon. Flavor enhancers include, and are not limited to, citric acid.

Suitable suspending agents include, for example, pre-gelatinized starch, powdered cellulose, microcrystalline cellulose, methylcellulose, ethylmethylcellulose, ethylcellulose, sodium carboxymethylcellulose, hydroxyethylcellulose, hydroxypropylmethylcellulose, ethylhydroxyethylcellulose, hydroxypropylcellulose, attapulgile (colloidal magnesium aluminum silicate), bentonite (colloidal aluminum silicate), hectorite (colloidal magnesium aluminum silicate), sepiolite (magnesium silicate), magnesium aluminum silicate, silica gel, colloidal silicon dioxide, acacia, agar, carrageenan, guar gum, karaya gum, locust bean gum, pectin, sodium alginate, propylene glycol alginate, tamarind gum, tragacanth, xanthan gum, carbomer, povidone, polyethylene glycols, gelatin, glycyrrhizin and sodium starch glycolate.

Suitable sustained release coatings agents include ethylcellulose, polymethacrylates, and the like.

Additional conventional excipients, which may be added, include preservatives, stabilizers, anti-oxidants, silica flow conditioners, antiadherents or glidants.

Preservatives include but are not limited to sodium benzoate, potassium sorbate, salts of edetate (also known as salts of ethylenediaminetetraacetic acid, or EDTA, such as disodium edetate), parabens (such as methyl, ethyl, propyl and butyl p-hydroxybenzoic acids esters or mixtures thereof) or mixtures thereof.

Other suitable fillers, binders, disintegrants, lubricants and other excipients which may be used are described in Handbook of Pharmaceutical Excipients, 2^{nd} Edition, American Lachman, Leon, 1976; Pharmaceutical Dosage Forms: Tablets Volume 1, 2^{nd} Edition, Lieberman, Herbert A., et al, 1989; Modern Pharmaceutics, Banker, Gilbert and Rhodes, Christopher T, 1979; and Remington's Pharmaceutical Sciences, 15^{th} Edition, 1975, each of which is incorporated herein by reference in its entirety.

The solid formulations of the invention can include, for example, about 0.1 to about 99%, about 0.1 to about 90, about 0.1 to about 85, about 0.1 to about 80, about 0.1 to about 75, about 0.1 to about 70, about 0.1 to about 65, about 0.1 to about 60, about 0.1 to about 55, about 0.1 to about 50, about 0.1 to about 45, about 0.1 to about 40, about 0.1 to about 35, about 0.1 to about 30, about 0.1 to about 25, about 0.1 to about 20, about 0.1 to about 15, about 0.1 to about 12, about 0.1 to about 10, about 0.1 to about 8, about 0.1 to about 5, about 0.1 to about 4, about 0.1 to about 3, about 0.1 to about 2, about 0.1 to about 1.5, about 0.1 to about 1, or about 0.1 to about 0.5% by weight of 11-piperazin-1-yldibenzo[b,f][1,4]thiazepine (Formula I). In some embodiments, the solid formulation contains about 0.1 to about 0.3, about 0.7 to about 2.0, about 4.0 to about 10.0, about 14.0 to about 37.0, or about 40.0 to about 60.0% by weight of 11-piperazin-1-yldibenzo[b,f][1,4]thiazepine (Formula I). In some embodiments, the solid formulation contains about 0.2, about 0.8, about 1.0, about 1.7, about 5.0, about 8.3, about 10.0, about 16.7, about 25.0, about 33.3, or about 50.0% by weight of 11-piperazin-1-yldibenzo[b,f][1,4]thiazepine (Formula I).

The solid formulations of the invention can further include a filler. Suitable fillers are provided herein above. In some embodiments, the filler includes an inorganic salt such as alkali metal or alkaline earth metal salts of chloride, phosphates, sulfates, and the like. In some embodiments, the filler contains dibasic calcium phosphate dihydrate. In some embodiments, the inorganic filler is present in an amount of about 1 to about 25, about 1 to about 20, or about 3 to about 17% by weight. In some embodiments, the inorganic filler is present in an amount of about 3, about 4, about 5, about 6, about 7, about 8, about 10, about 11, about 12, about 13, about 14, about 15, about 16, or about 17% by weight.

In some embodiments, the formulations of the invention include a cellulose filler such as microcrystalline cellulose or silicified microcrystalline cellulose. The cellulose filled can be present in an amount of about 10 to about 95, about 10 to about 75, about 10 to about 60, about 15 to about 50, about 40 to about 90, or about 50 to about 90% by weight of a cellulose filler. In some embodiments, the microcrystalline cellulose is present in an amount of about 15 to about 50% by weight. In some embodiments, silicifed microcrystalline cellulose is present in an amount of about 50 to about 90% by weight. In some embodiments, the cellulose is present in an amount of about 15, about 16, about 17, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 53, about 54, about 55, about 76, about 77, about 78, about 81, about 82, about 83, about 84, about 85, or about 86% by weight.

In some embodiments, the present formulations include an oligosaccharide filler such as lactose. In some embodiments, the oligosaccharide filler is present in an amount of about 15 to about 50, about 15 to about 45, or about 18 to about 43% by weight. In some embodiments, the oligosaccharide filler is present in an amount of about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, or about 44% by weight.

In some embodiments, formulations contain a non-cellulosic binder such as povidone or copovidone. In some embodiments, the binder is present in an amount of about 0.5 to about 15, about 0.5 to about 10, or about 1 to about 10% by weight. In some embodiments, the binder is present in an amount of about 1, about 2, about 3, about 4, about 5, about 6, about 7, about 8, about 9, or about 10% by weight.

In some embodiments, the formulations contain a disintegrant such as sodium starch glycolate or crospovidone. For example, the disintegrant is present in an amount of about 1 to about 15, about 1 to about 12, or about 1 to about 10% by weight of a disintegrant. In some embodiments, the formulations contain about 1, about 2, about 3, about 4, about 5, about 6, about 7, about 8, about 9, or about 10% by weight of disintegrant.

In some embodiments, the formulations of the invention contain a lubricant such as magnesium stearate or sodium stearyl fumarate. For example, the lubricant can be present in an amount of about 0.1 to about 8, about 0.5 to about 6, about 0.5 to about 5, or about 0.5 to about 3% by weight. In some embodiments, the lubricant can be present in an amount of about 0.5, about 0.75, about 1, about 1.5, about 2, about 3, about 4, or about 5% by weight.

In some embodiments, the formulation contains a suspending agent. The suspending agent can be present in an amount of about 1 to about 10%, about 1 to abut 8%, or about 1 to about 5% by weight. In some embodiments, the suspending agent is present in an amount of about 4, about 5, or about 6% by weight.

In some embodiments, the formulation contain a coating agent. The coating agent can be present in an amount of about 1 to about 15%, about 1 to about 10%, about 1 to abut 8%, or about 1 to about 5% by weight. In some embodiments, the coating agent is present in an amount of about 9, about 10, or about 11% by weight.

In some embodiments, the solid formulation contains about 0.1 to about 75% by weight of 11-piperazin-1-yldibenzo[b,f][1,4]thiazepine; about 1 to about 25% by weight of an inorganic salt filler; about 10 to about 95% by weight of a cellulose filler; about 15 to about 50% by weight of an oligosaccharide filler; about 0.5 to about 15% by weight of a non-cellulosic binder; about 1 to about 15% by weight of a disintegrant; and about 0.1 to about 8% by weight of a lubricant.

In further embodiments, the solid formulation contains about 0.1 to about 75% by weight of said 11-piperazin-1-yldibenzo[b,f][1,4]thiazepine; about 1 to about 25% by weight of dibasic calcium phosphate dihydrate; about 10 to about 95% by weight of microcrystalline cellulose or silicified microcrystalline cellulose; about 15 to about 50% by weight of lactose; about 0.5 to about 15% by weight of povidone or copovidone; about 1 to about 15% by weight of sodium starch glycolate or crospovidone; and about 0.1 to about 8% by weight of magnesium stearate or sodium stearyl fumarate.

In further embodiments, the solid formulation contains about 0.1 to about 75% by weight of 11-piperazin-1-yldibenzo[b,f][1,4]thiazepine; about 40 to about 90% by weight of a cellulose filler; about 1 to about 15% by weight of a disintegrant; and about 0.1 to about 8% by weight of a lubricant.

In further embodiments, the solid formulation contains about 0.1 to about 75% by weight of 11-piperazin-1-yldibenzo[b,f][1,4]thiazepine; about 40 to about 90% by weight of silicified microcrystalline cellulose; about 1 to about 15% by weight of crospovidone; and about 0.1 to about 8% by weight of sodium stearyl fumarate.

In further embodiments, the solid formulation contains about 0.1 to about 75% by weight of 11-piperazin-1-yldibenzo[b,f][1,4]thiazepine; about 25 to about 55% by weight of a cellulose filler; about 15 to about 50% by weight of an oligosaccharide filler; about 1 to about 15% by weight of a disintegrant; and about 0.1 to about 8% by weight of a lubricant.

In further embodiments, the solid formulation contains about 0.1 to about 75% by weight of 11-piperazin-1-yldibenzo[b,f][1,4]thiazepine; about 25 to about 55% by weight of microcrystalline cellulose; about 15 to about 50% by weight of lactose; about 1 to about 15% by weight of crospovidone; and about 0.1 to about 8% by weight of a magnesium stearate.

The solid formulations of the invention can be used to prepare solid dosage forms such as tablets, caplets, capsules, sachets, and the like. In some embodiments, the solid dosage form is suitable for oral administration. The amount of 11-piperazin-1-yldibenzo[b,f][1,4]thiazepine in a solid dosage form can be about 0.1 to about 1000 mg, about 0.1 to about 750 mg, or about 0.1 to about 500 mg. In some embodiments, the amount of 11-piperazin-1-yldibenzo[b,f][1,4]thiazepine present in a solid dosage form is about 1, about 2, about 3, about 4, about 5, about 10, about 25, about 50, about 100, about 200, about 300, about 400, about 500, or about 600 mg. In some embodiments, the total weight (e.g., active ingredients plus excipients, coatings, etc.) of the solid dosage form is about 50 to about 1500 mg. For example, the total weight of the solid dosage form is about 100, about 200, about 300, about 400, about 500, about 600, about 700, about 800, about 900, about 1000, about 1100, or about 1200 mg.

The active ingredient 11-piperazin-1-yldibenzo[b,f][1,4]thiazepine can be formulated as a powder dosage form suitable for preparation of a suspension just prior to use or alternatively suitable for addition to food. The formulation is typically a free flowing powder with a light bulk density. This formulation can be prepared by using a combination of excipients including, for example, a filler, a sweetener, and a suspending agent. Examples of fillers include lactose, starch, maltodextrin, hypromellose, microcrystalline cellulose, and the like. Examples of sweeteners include aspartame, lactitol, sacchrin, sucrose, fructose, xylitol, and the like. Examples of suspending agents include carboxymethylcellulose calcium, xantham gum, ceraonia, saponite, maltitol, hypromellose, colloidal silicon dioxide, and the like.

The solid formulations of the invention can include, in addition to 11-piperazin-1-yldibenzo[b,f][1,4]thiazepine, a further active ingredient. Example further active ingredients include benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, serotonin reuptake inhibitors or a mood stabilizer.

Exemplary benzodiazepines include but are not limited to adinazolam, alprazolam, bromazepam, clonazepam, chlorazepate, chlordiazepoxide, diazepam, estazolam, flurazepam, balezepam, lorazepam, midazolam, nitrazepam, oxazepam, quazepam, temazepam, triazolam and equivalents thereof.

Exemplary 5-HT_{1A} and/or 5HT_{1B} ligands include but are not limited to buspirone, alnespirone, elzasonan, ipsapirone, gepirone, zopiclone and equivalents thereof.

Exemplary mGluR2 agonists may include (1S,3R)-1-aminocyclopentane-1,3-dicarboxylic acid, (2S,3S,4S)alpha-(carboxycyclopropyl)glycine, and 3,5-dihydroxyphenylglycine.

Exemplary antidepressants include but are not limited to maprotiline, amitriptyline, clomipramine, desipramine, doxepin, imipramine, nortryptyline, protriptyline, trimipramine, SSRIs and SNRIs such as fluoxetine, paroxetine, citalopram, escitalopram, sertraline, venlafaxine, fluoxamine, and reboxetine.

Exemplary antipsychotics include but are not limited to clozapine, risperidone, quetiapine, olanzapine, amisulpride, sulpiride, zotepine, chlorpromazine, haloperidol, ziprasidone, and sertindole.

Exemplary mood stabilizers may include but are not limited to Valproic acid (valproate) and its derivative (e.g. divalproex), lamotrigine, lithium, verapamil, carbamazepine and gabapentin.

The formulations of the invention can be obtained by conventional procedures using conventional techniques. For example, the active ingredient is typically mixed with a solid excipient, diluted by a solid excipient or enclosed within such a carrier in the form of, for example, a capsule, sachet, paper, or other container. Thus, the compositions can be in the form of tablets, pills, powders, lozenges, sachets, cachets, ointments containing, for example, up to 10% by weight of the active compound, soft and hard gelatin capsules, suppositories, and sterile packaged powders.

The formulation of the invention can be administered by any route including orally, subcutaneously, topically, and the like.

The amount of active ingredient that is combined with one or more solid excipients to produce a single dosage form will necessarily vary depending upon the host treated and the particular route of administration. The size of the dose for therapeutic or prophylactic purposes of the active compound(s) will naturally vary according to the nature and severity of the symptoms or conditions, the age and sex of the animal or patient and the route of administration, according to well known principles of medicine.

The present invention further provides methods of treating at least one symptom or condition associated with schizophrenia and other psychotic disorders (e.g., psychotic disorder, psychosis); dementia and other cognitive disorders, anxiety disorders (e.g., generalized anxiety disorder); mood disorders (e.g., depressive disorders, major depressive disorders; bipolar disorders including bipolar I and II, bipolar mania, bipolar depression); sleep disorders; disorders usually first diagnosed in infancy, childhood, or adolescence (e.g., attention-deficit disorder and disruptive behavior disorders); and neurodegenerative disorders comprising administering to a mammal a pharmaceutically effective amount of a solid formulation of the invention or composition containing one or more of the same. In some embodiments, the symptoms and conditions include but are not limited to anxiety, agitation, hostility, panic, eating disorders, affective symptoms, mood symptoms, negative and positive psychotic symptoms commonly associated with psychosis and neurodegenerative disorders. In some embodiments, the symptoms and conditions are any of psychosis, schizophrenia, bipolar I, and anxiety.

In some embodiments, the present invention further provides methods of treating at least one symptom or condition associated with but not limited to: 1) Schizophrenia and other Psychotic Disorders including but not limited to Psychotic Disorder, Schizophreniform Disorder, Schizoaffective Disorder, Delusional Disorder, Brief Psychotic Disorder, Shared Psychotic Disorder, and Psychotic Disorder Due to a General Medical Condition; 2) Dementia and other Cognitive Disorders; 3) Anxiety Disorders including but not limited to Panic Disorder Without Agoraphobia, Panic Disorder With Agoraphobia, Agoraphobia Without History of Panic Disorder, Specific Phobia, Social Phobia, Obsessive-Compulsive Disorder, Postraumatic Stress Disorder, Acute Stress Disorder, Generalized Anxiety Disorder and Generalized Anxiety Disorder Due to a General Medical Condition; 4) Mood Disorders including but not limited to a) Depressive Disorders, including but not limited to Major Depressive Disorder and Dysthymic Disorder and b) Bipolar Depression and/or Bipolar mania including but not limited to Bipolar I Disorder, including but not limited to those with manic, depressive or mixed episodes, and Bipolar II Disorder, c) Cyclothymic Disorder, d) Mood Disorder Due to a General Medical Condition; 5) Sleep Disorders; 6) Disorders Usually First Diagnosed in Infancy, Childhood, or Adolescence including but not limited to Mental Retardation, Learning Disorders, Motor Skills Disorder, Communication Disorders, Pervasive Developmental Disorders, Attention-Deficit and Disruptive Behavior Disorders, Feeding and Eating Disorders of Infancy or Early Childhood, Tic Disorders, and Elimination Disorders; 7) Substance-Related Disorders including but not limited to Substance Dependence, Substance Abuse, Substance Intoxication, Substance Withdrawal, Alcohol-Related Disorders, Amphetamine (or Amphetamine-Like)-Related Disorders, Caffeine-Related Disorders, Cannabis-Related Disorders, Cocaine-Related Disorders, Hallucinogen-Related Disorders, Inhalant-Related Disorders, Nicotine-Related Disorders, Opioid-Related Disorders, Phencyclidine (or Phencyclidine-Like)-Related Disorders, and Sedative-, Hypnotic- or Anxiolytic-Related Disorders; 8) Attention-Deficit and Disruptive Behavior Disorders; 9) Eating Disorders; 10) Personality Disorders including but not limited to Obsessive-Compulsive Personality Disorder; and 11) Impulse-Control Disorders, by administering to a patient a pharmaceutically effective amount of a formulation described herein.

The above conditions and disorders are defined for example in the American Psychiatric Association: Diagnostic and Statistical Manual of Mental Disorders, Fourth Edition, Text Revision, Washington, D.C., American Psychiatric Association, 2000. Substance abuse and substance dependence and related disorders are also defined therein. This Manual may also be referred to for greater detail on the symptoms and diagnostic features associated with substance use, abuse and dependence. Typical substances that lead to substance abuse and substance dependence include drugs such as amphetamines, cannabis, cocaine, crack, hallucinogenic agents, inhalants, opioids, phencyclidine, sedatives, hypnotics, anxiolytic agents and alcohol. Nicotine can also lead to substance dependence.

In some embodiments, the symptoms and conditions that may be treated using an effective amount of a solid formulation of the invention include Depressive Disorders (e.g., Major Depressive Disorder), Anxiety Disorders (e.g., Generalized Anxiety Disorder), and Substance-Related Disorders.

The present invention further provides methods of treating at least one symptom or condition described herein by administering to a mammal a pharmaceutically effective amount of a solid formulation of the invention and a therapeutically effective amount of at least one other therapeutically active agent selected from benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, serotonin reuptake inhibitors, and mood stabilizers.

Administration of two or more active agents can be carried out in combination, e.g., as part of the same formulation, or separately (e.g., serially or consecutively) as part of an appropriate dose regimen designed to obtain the benefits of combination therapy. The appropriate dose regimen, the amount of each dose of an active agent administered, and the specific intervals between doses of each active agent will depend upon the subject being treated, the specific active agent being administered and the nature and severity of the specific disorder or condition being treated.

In general, the formulations provided herein can be administered to a mammal in an amount up to about 750 mg of active agent per day, particularly from about 75 mg to about 750 mg per day, in single or divided doses. In another aspect of the invention, the formulations provided herein may be administered to a mammal in an amount from about 1 mg to about 600 mg per day. In a further aspect of the invention, the formulations provided herein may be administered in an amount from about 100 mg to about 400 mg per day. The formulation may be administered on a regimen of up to 6 times per day, or 1 to 4 times per day. Variations can occur depending upon the mammal being treated and the individual response to the treatment, as well as on the type of pharmaceutical formulation chosen and the time period and interval at which such administration is carried out. In some instances, dosage levels below the lower limit of the aforesaid range may be more than adequate, while in other cases larger doses may be employed to achieve the desired effect, provided that such larger doses are first divided into several small doses for administration throughout the day.

In some embodiments, the formulation is administered comprising a predetermined dosage to a mammal between one and four times a day, wherein the predetermined dosage is from about 1 mg to about 600 mg.

The present invention also provides a method of treating the symptoms or conditions provided herein comprising the step of administering an initial predetermined dosage of the active ingredient in a formulation of the invention to a human patient twice a day, wherein the predetermined dosage is between 1 mg and 30 mg with increases in increments of 1-50 mg twice daily on the second and third day as tolerated. Thereafter, further dosage adjustments can be made at intervals of 2 days or greater.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “therapeutically effective amount” refers to amount of active compound or pharmaceutical agent that elicits the biological or medicinal response in a tissue, system, animal, individual or human that is being sought by a researcher, veterinarian, medical doctor or other clinician and can be readily determined by a clinician by using numerous methods already known in the art, an example of which is the BPRS cluster score that can be used to assess levels of hostility and positive symptoms.

The term “treating” within the context of the present invention is meant to encompass the administration a therapeutically effective amount of the compound of Formula I to mitigate or inhibit either a pre-existing disease state, acute or chronic, or a recurring symptom or condition. Also encompassed are prophylactic therapies for prevention of recurring conditions and continued therapy for chronic disorders.

The term “mammal” is meant to refer to any warm-blooded animal, preferably a human. In some embodiments, the mammal is in need of treatment because it is suffering from or prone to developing one or more of the symptoms, diseases or disorders described above.

Any or all of the solid formulations described herein, including any combination thereof, can be used in the preparation of a medicament for the treatment of any of the diseases, disorders, or conditions described herein.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of 11-piperazin-1-yldibenzo[b,f][1,4]thiazepine

### Preparation A (Form A)

Aqueous solution (584 mL; e.g., prepared by extraction of 11-piperazin-1-yldibenzo[b,f][1,4]thiazepine into water/HCl from a toluene solution such as described below in Preparation B) containing 11-piperazin-1-yldibenzo[b,f][1,4]thiazepine hydrochloride was charged to a jacketed 1 L flask. The flask was then charged with toluene (500 mL) and sodium hydroxide (48% w/w, 33.0 g). The mixture was stirred at 70° C. for 30 minutes and became white and cloudy. The mixture was then allowed to settle for 30 min and the phases were separated. The toluene layer was washed at 70° C. with 2×100 mL of water (1^{st} wash=pH 10.3; 2^{nd} wash=pH 8.0). The final toluene volume was 560 mL containing about 74 g of 11-piperazin-1-yldibenzo[b,f][1,4]thiazepine in good purity.

The above procedure was repeated for an additional four aqueous solutions of 11-piperazin-1-yldibenzo[b,f][1,4]thiazepine hydrochloride and the five resulting toluene solutions were combined and evaporated to dryness on a rotary evaporator. The resulting hard solid was then charged to a jacketed vessel and slurried with methyl-t-butyl ether (MTBE) (500 mL). The resulting slurry was stirred overnight at ambient temperature and then cooled to 5° C. and held for 4 h. The solid 11-piperazin-1-yldibenzo[b,f][1,4]thiazepine product was isolated on a no. 3 sinter and washed with 200 mL of cold MTBE. The cake was dried in a vacuum oven overnight at 60° C. yielding 373 g of product.

### Preparation B (Form A)

A toluene solution of 11-piperazin-1-yldibenzo[b,f][1,4]thiazepine (1500 mL, 0.686 mol) prepared by reaction of piperazine with 11-chloro-dibenzo[b,f][1,4]-thiazepine in toluene (see, e.g., U.S. Pat. No. 4,879,288) was treated with 1500 mL deionized water and 90 mL of HCl (32% w/w). The resulting mixture was heated to 70° C. and agitated for 45 min. Agitation was ceased and the mixture allowed to settle and phase separate for 30 min. The lower aqueous phase, containing the HCl salt of 11-piperazin-1-yldibenzo[b,f][1,4]thiazepine was isolated. The aqueous phase was then treated with 1000 mL of toluene and 99 g of aqueous NaOH (47% w/w). The resulting mixture was heated to 70° C. and agitated for 45 min. Agitation was ceased and the mixture allowed to settle and phase separate for 30 min. The lower aqueous phase was discarded and the upper organic phase retained to which 300 mL of deionized water was added. The resulting mixture was agitated for 15 min and then allowed to settle for 30 min. The aqueous phase was discarded and the organic phase retained. The organic phase was extracted once more with 300 mL of deionized water. About 750 mL of toluene from the organic phase was distilled out. The resulting concentrate was cooled to 60° C., then 200 mL of methyl-t-butyl ether (MTBE) was added. The resulting mixture was cooled to ambient temperature then seeded with Form A seed crystals. The seeded mixture was then cooled to 10° C. and held at this temperature for 3 hours under slow agitation. The resulting solid was isolated under suction via a no. 3 sinter. The solid product was then washed with 120 mL of MTBE at ambient temperature and dried at 40° C. under vacuum resulting in 175 g (86.4%) of crystalline product. Assay 99.9% w/w by HPLC area %.

Solid 11-piperazin-1-yldibenzo[b,f][1,4]thiazepine (30 g, 0.1016 mol) prepared as described above was slurried in isopropanol (120 mL). The resulting mixture was warmed to about 63-64° C. to completely dissolve the solid. The resulting solution was filtered through a preheated (about 55° C.) split Buchner funnel fitted with filter paper with a pore size of 6 □m. The filtered solution was then adjusted to 55° C. and seeded with seed crystals of Form A (0.024 g). The seeded solution was maintained at 55° C. for about 2 h then linearly cooled to 40° C. over the course of 6 h, linearly cooled to 20° C. over the course of 2 h, and then linearly cooled to 0° C. over the course of 1 h. The resulting slurry was held at 0° C. for 12 h and the solid product cake (13 mm high×68 mm diameter) was isolated by filtration. The product cake was displacement washed with 30 mL isopropanol prechilled to 0° C. and the cake allowed to deliquor. The product was then dried at 40° C. under vacuum yielding 24.9 g (83%) of Form A. Assay by NMR: 98.9% w/w.

X-ray powder diffraction peak data of crystalline Form A is provided below in Chart A.

CHART A
(Form A)

| Angle | Intensity | |
|---|---|---|
| 2-Theta ° | Count | Intensity % |
| 10.8 | 18321 | 51.4 |
| 12.3 | 2390 | 6.7 |
| 13.3 | 24555 | 68.9 |
| 15.2 | 12193 | 34.2 |
| 15.3 | 9799 | 27.5 |
| 16.0 | 2414 | 6.8 |
| 17.2 | 18803 | 52.7 |
| 18.8 | 6502 | 18.2 |
| 19.3 | 7290 | 20.4 |
| 20.0 | 3666 | 10.3 |
| 20.4 | 15535 | 43.6 |
| 21.2 | 25874 | 72.6 |
| 21.7 | 16902 | 47.4 |
| 22.1 | 1473 | 4.1 |
| 24.1 | 3968 | 11.1 |
| 24.2 | 2197 | 6.2 |
| 24.9 | 3579 | 10 |
| 25.5 | 35663 | 100 |
| 26.4 | 6298 | 17.7 |
| 27.9 | 3290 | 9.2 |
| 28.0 | 3746 | 10.5 |
| 28.3 | 2206 | 6.2 |
| 28.6 | 2711 | 7.6 |
| 28.9 | 2142 | 6 |
| 29.4 | 4006 | 11.2 |
| 29.8 | 2464 | 6.9 |
| 30.4 | 2754 | 7.7 |
| 30.9 | 5213 | 14.6 |
| 31.0 | 5143 | 14.4 |
| 31.6 | 2053 | 5.8 |
| 32.1 | 3643 | 10.2 |
| 32.4 | 4234 | 11.9 |
| 32.5 | 3827 | 10.7 |
| 33.2 | 2102 | 5.9 |
| 34.6 | 1540 | 4.3 |
| 35.8 | 1543 | 4.3 |
| 36.3 | 3768 | 10.6 |
| 36.9 | 3086 | 8.7 |
| 38.1 | 2062 | 5.8 |
| 39.0 | 2801 | 7.9 |
| 39.4 | 1492 | 4.2 |

### Preparation C (Amorphous)

Into a 1000 mL round-bottom flask equipped with a magnetic stirring bar and reflux condenser with a nitrogen inlet was charged with 25.0 g (0.110 mol) of dibenzo[b,f][1,4]thiazepine-11(10-H)-one (made by the method of J. Schmutz et al. Helv. Chim. Acta., 48: 336 (1965)), as a dry solid, followed by 310 mL POCl₃ and 3 mL of N,N-dimethylaniline. The reaction mixture was heated at reflux (106° C.) for 6 hours giving a clear orange solution. The reaction was then cooled to room temperature, and POCl₃ removed on the rotary evaporator leaving an orange oil. This residue was partitioned between ice-water (500 mL) and ethyl acetate (800 mL). The layers were separated and the aqueous phase extracted with ethyl acetate (3×200 mL). The combined ethyl acetate extracts were dried over MgSO₄, filtered, and then stripped down on the rotary evaporator, leaving the crude imino chloride as a light yellow solid (26.26 g, 97% yield). The structure was confirmed by NMR and mass spectroscopy (300 MHz, CDCl₃; ES+, M+1=246.7). Crude imino chloride (27.35 g, 0.111 mol) was added to 1000 mL o-xylene in a 2000 mL round-bottom flask equipped with a magnetic stir bar and a reflux condenser with nitrogen inlet. To this solution was added commercially available piperazine (47.95 g, 0.557 mol) in one portion as a dry solid at room temperature. The mixture was stirred until nearly all the piperazine dissolved. Then the reaction mixture was heated at reflux (142° C.) for 40 hours (out of convenience). The reaction was then allowed to cool to room temperature, and an aliquot was partitioned between 1 N NaOH /CH₂Cl₂. The organic phase was checked by TLC (silica gel, CH₂Cl₂/methanol 90:10, iodoplatinate visualized) and showed clean conversion to one major product (Rf=0.45). A drop of the reaction solution was diluted with CH₃CN to prepare a sample for LC/MS analysis, which confirmed the presence of the desired product (M+1=296.4). The reaction mixture was stripped down on the rotary evaporator under high vacuum to remove the xylene. The residue was partitioned between 1 N NaOH (400 mL) and CH₂Cl₂ (200 mL). The layers were separated, and the aqueous phase further extracted with CH₂Cl₂ (3×200 mL). The combined CH₂Cl₂ extracts were washed with brine (200 mL), then dried over MgSO₄, filtered, and stripped down on the rotary evaporator to give the crude title compound as a yellow gum (35.3 g). The crude free base was purified by flash column chromatography over silica gel (600 g) eluting with a gradient of 0 to 20% methanol in CH₂Cl₂. Fractions containing the pure desired product were combined and stripped down on the rotary evaporator, to afford the purified free base as a light, yellow foam (25.67 g, 78% yield).

### Example 2

### Stability of Form A

Individual samples of Form A were slurried in various solvents (acetone, ethanol, ethyl acetate, methylethyl ketone, toluene, and water). The mixtures were stirred overnight at room temperature in sealed containers. The samples were then filtered and vacuum dried at 50° C. for 2 h. The resulting material in each of the solvents tested was a white crystalline material having an XRPD diffraction pattern consistent with Form A. Accordingly, Form A has good stability in a variety of solvents and workup conditions.

### Example 3

### Thermal Analysis of Form A

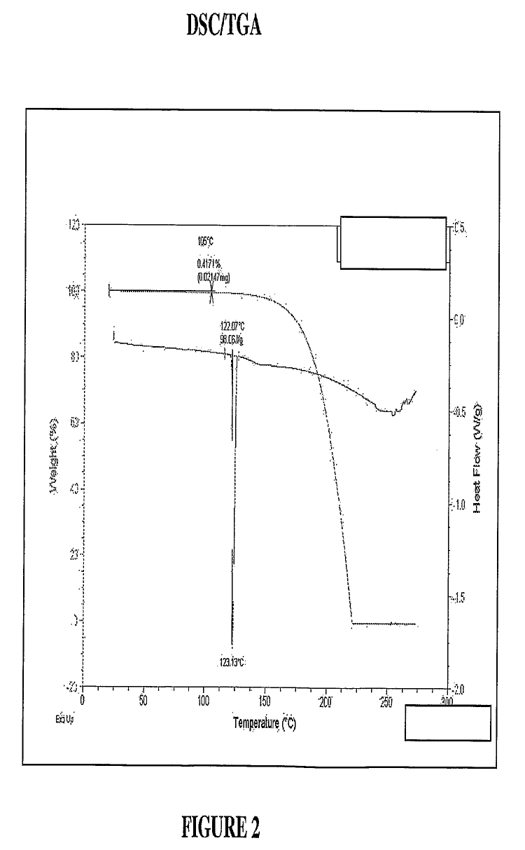

DSC and TGA data consistent with Form A are provided in FIG. 2. The DSC data displayed one sharp endothermic event at 123.1° C. which corresponded to a melt event prior to degradation. The TGA data shows 0.4% weight loss in the water/solvent region.

### Example 4

### Dynamic Vapor Sorption Analysis of Form A

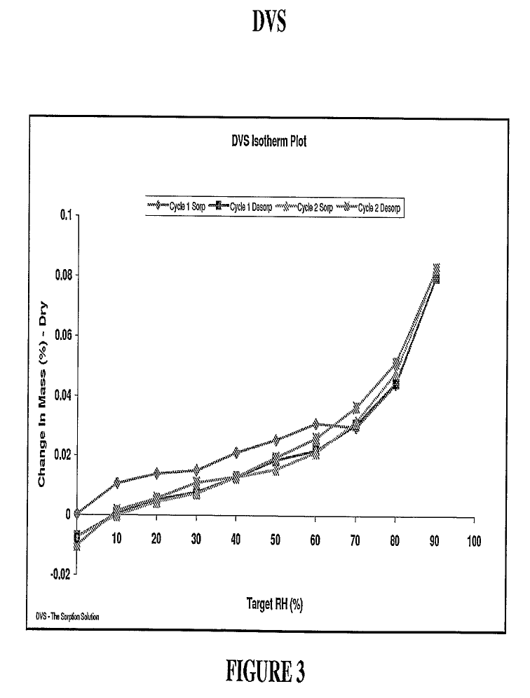

DVS data of Form A revealed that the crystalline form is non-hygroscopic showing only slight, reversible water gain without hysteresis. As shown in FIG. 3, two cycles overlay well with no evidence of form change.

### Example 5

### Wet Granulation Formulations

Solid 11-piperazin-1-yldibenzo[b,f][1,4]thiazepine, povidone, dibasic calcium phosphate, and a portion of the microcrystalline cellulose and sodium starch glycolate are mixed with water in a granulator to form granules. The granules are dried in a dryer and then sized used using a mill fitted with the appropriate screen. To the milled material, the remainder of the microcrystalline cellulose and sodium starch glycolate along with lactose is added and blended. To this, the magnesium stearate is added and blended further. This mixture is compressed into tablets using a tablet compression machine or filled into capsules using an encapsulation device or filled into sachets. Example wet granulation formulations are provided in the tables below.

TABLE A

| Strength | 1 mg | 5 mg | 10 mg | 50 mg | 100 mg | 200 mg | 300 mg |
|---|---|---|---|---|---|---|---|
| Formula I (mg) | 1.00 | 5.00 | 10.00 | 50.00 | 100.00 | 200.00 | 300.00 |
| Povidone (mg) | 6.25 | 6.25 | 6.25 | 12.50 | 25.00 | 17.00 | 25.00 |
| Dibasic calcium phosphate | 25.00 | 25.00 | 25.00 | 50.00 | 100.00 | 20.00 | 30.00 |
| dihydrate (mg) | | | | | | | |
| Microcrystalline cellulose | 283.25 | 280.39 | 277.53 | 251.93 | 212.87 | 161.74 | 98.61 |
| (mg) | | | | | | | |
| Lactose (mg) | 258.00 | 256.86 | 254.72 | 201.57 | 128.13 | 167.26 | 112.39 |
| Sodium starch glycolate | 17.50 | 17.50 | 17.50 | 25.00 | 25.00 | 25.00 | 25.00 |
| (mg) | | | | | | | |
| Magnesium stearate (mg) | 9.00 | 9.00 | 9.00 | 9.00 | 9.00 | 9.00 | 9.00 |
| Total (mg) | 600.00 | 600.00 | 600.00 | 600.00 | 600.00 | 600.00 | 600.00 |

TABLE B

| Strength | 1 mg |
|---|---|
| Formula I (mg) | 1.00 |
| Povidone (mg) | 3.32-9.29 |
| Dibasic calcium phosphate dihydrate (mg) | 10.61-13.27 |
| Microcrystalline cellulose (mg) | 37.05-42.74 |
| Lactose (mg) | 25.06-35.52 |
| Sodium starch glycolate (mg) | 5.31-9.29 |
| Magnesium stearate (mg) | 0.75-1.50 |
| Total (mg) | 100.00 |

TABLE C

| Strength | 5 mg |
|---|---|
| Formula I (mg) | 5.00 |
| Povidone (mg) | 3.18-8.90 |
| Dibasic calcium phosphate dihydrate (mg) | 10.18-12.72 |
| Microcrystalline cellulose (mg) | 35.54-40.99 |
| Lactose (mg) | 28.98-34.07 |
| Sodium starch glycolate (mg) | 5.09-8.90 |
| Magnesium stearate (mg) | 0.75-1.50 |
| Total (mg) | 100.00 |

TABLE D

| Strength | 10 mg |
|---|---|
| Formula I (mg) | 10.00 |
| Povidone (mg) | 3.01-8.43 |
| Dibasic calcium phosphate dihydrate (mg) | 9.63-12.04 |
| Microcrystalline cellulose (mg) | 33.71-42.28 |
| Lactose (mg) | 22.75-32.25 |
| Sodium starch glycolate (mg) | 4.82-8.43 |
| Magnesium stearate (mg) | 0.75-1.50 |
| Total (mg) | 100.00 |

TABLE E

| Strength | 25 mg |
|---|---|
| Formula I (mg) | 25.00 |
| Povidone (mg) | 2.50-7.00 |
| Dibasic calcium phosphate dihydrate (mg) | 8.00-10.00 |
| Microcrystalline cellulose (mg) | 28.00-35.11 |
| Lactose (mg) | 18.89-26.78 |
| Sodium starch glycolate (mg) | 4.00-7.00 |
| Magnesium stearate (mg) | 0.75-1.50 |
| Total (mg) | 100.00 |

TABLE F

| Strength | 50 mg |
|---|---|
| Formula I (mg) | 50.00 |
| Povidone (mg) | 5.00-14.00 |
| Dibasic calcium phosphate dihydrate (mg) | 16.00-20.00 |
| Microcrystalline cellulose (mg) | 56.00-70.22 |
| Lactose (mg) | 37.78-53.56 |
| Sodium starch glycolate (mg) | 8.00-14.00 |
| Magnesium stearate (mg) | 1.50-3.00 |
| Total (mg) | 200.00 |

TABLE G

| Strength | 100 mg |
|---|---|
| Formula I (mg) | 100.00 |
| Povidone (mg) | 10.00-28.00 |
| Dibasic calcium phosphate dihydrate (mg) | 32.00-40.00 |
| Microcrystalline cellulose (mg) | 112.00-140.44 |
| Lactose (mg) | 75.56-107.12 |
| Sodium starch glycolate (mg) | 16.00-28.00 |
| Magnesium stearate (mg) | 6.00 |
| Total (mg) | 400.00 |

TABLE H

| Strength | 200 mg |
|---|---|
| Formula I (mg) | 200.00 |
| Povidone (mg) | 20.00-56.00 |
| Dibasic calcium phosphate dihydrate (mg) | 64.00-80.00 |
| Microcrystalline cellulose (mg) | 224.00-280.88 |
| Lactose (mg) | 151.12-214.24 |
| Sodium starch glycolate (mg) | 32.00-56.00 |
| Magnesium stearate (mg) | 6.00-12.00 |
| Total (mg) | 800.00 |

TABLE I

| Strength | 300 mg |
|---|---|
| Formula I (mg) | 300.00 |
| Povidone (mg) | 30.00-84.00 |
| Dibasic calcium phosphate dihydrate (mg) | 96.00-120.00 |
| Microcrystalline cellulose (mg) | 336.00-421.32 |
| Lactose (mg) | 226.68-321.36 |
| Sodium starch glycolate (mg) | 48.00-84.00 |
| Magnesium stearate (mg) | 9.00-18.00 |
| Total (mg) | 1200.00 |

TABLE J

| Strength | 1 mg | 5 mg | 10 mg | 25 mg | 50 mg | 100 mg |
|---|---|---|---|---|---|---|
| Formula I (mg) | 1.00 | 5.00 | 10.00 | 25.00 | 50.00 | 100.00 |
| Povidone (mg) | 8.37 | 8.03 | 7.60 | 6.31 | 4.17 | 8.33 |
| Dibasic cal- | 10.05 | 9.64 | 9.12 | 7.58 | 5.00 | 10.00 |
| cium phosphate | | | | | | |
| dihydrate (mg) | | | | | | |
| Micro- | 33.05 | 31.69 | 30.00 | 24..92 | 16.44 | 32.88 |
| crystalline | | | | | | |
| cellulose (mg) | | | | | | |
| Lactose (mg) | 37.65 | 36.11 | 34.18 | 28.38 | 18.73 | 37.46 |
| Sodium starch | 8.37 | 8.03 | 7.60 | 6.31 | 4.17 | 8.33 |
| glycolate (mg) | | | | | | |
| Magnesium | 1.50 | 1.50 | 1.50 | 1.50 | 1.50 | 3.00 |
| stearate (mg) | | | | | | |
| Total (mg) | 100.00 | 100.00 | 100.00 | 100.00 | 100.00 | 200.00 |

TABLE K

| Strength | 200 mg | 300 mg | 400 mg | 500 mg | 600 mg |
|---|---|---|---|---|---|
| Formula I (mg) | 200.00 | 300.00 | 400.00 | 500.00 | 600.00 |
| Povidone (mg) | 16.66 | 24.99 | 33.32 | 41.65 | 49.98 |
| Dibasic calcium phos- | 20.00 | 30.00 | 40.00 | 50.00 | 60.00 |
| phate dihydrate (mg) | | | | | |
| Microcrystalline cell- | 65.76 | 98.64 | 131.52 | 164.40 | 197.28 |
| ulose (mg) | | | | | |
| Lactose (mg) | 74.92 | 112.38 | 149.84 | 187.30 | 224.76 |
| Sodium starch glyco- | 16.66 | 24.99 | 33.32 | 41.65 | 49.98 |
| late (mg) | | | | | |
| Magnesium stearate (mg) | 6.00 | 9.00 | 12.00 | 15.00 | 18.00 |
| Total (mg) | 400.00 | 600.00 | 800.00 | 1000.00 | 1200.00 |

TABLE L

| Strength | 1 mg | 5 mg | 10 mg | 25 mg | 50 mg | 100 mg | 200 mg | 300 mg |
|---|---|---|---|---|---|---|---|---|
| Formula I (mg) | 1.00 | 5.00 | 10.00 | 25.00 | 50.00 | 100.00 | 200.00 | 300.00 |
| Povidone (mg) | 9.27 | 8.89 | 8.42 | 7.00 | 14.00 | 28.00 | 56.00 | 83.62 |
| Dibasic calcium | 11.55 | 11.08 | 10.49 | 8.72 | 17.44 | 34.88 | 69.76 | 104.17 |
| phosphate | | | | | | | | |
| dihydrate (mg) | | | | | | | | |
| Microcrystalline | 37.08 | 35.57 | 33.68 | 28.00 | 56.00 | 112.00 | 224.00 | 334.49 |
| cellulose (mg) | | | | | | | | |
| Lactose (mg) | 30.83 | 29.57 | 28.00 | 23.28 | 46.56 | 93.12 | 186.24 | 278.10 |
| Sodium starch | 9.27 | 8.89 | 8.42 | 7.00 | 14.00 | 28.00 | 56.00 | 83.62 |
| glycolate (mg) | | | | | | | | |
| Magnesium | 1.00 | 1.00 | 1.00 | 1.00 | 2.00 | 4.00 | 8.00 | 16.00 |
| stearate (mg) | | | | | | | | |
| Total | 100.00 | 100.00 | 100.00 | 100.00 | 200.00 | 400.00 | 800.00 | 1200.00 |

### Example 6

### Direct Compression Formulations

An example compression formulation is provided in Table M.

11-Piperazin-1-yldibenzo[b,f][1,4]thiazepine and copovidone are blended. To this mixture, the dibasic calcium phosphate, microcrystalline cellulose, lactose, and sodium starch glycolate are added and blended further. The magnesium stearate is then added and blended further. This mixture is compressed into tablets using a tablet compression machine or filled into capsules using an encapsulation device or filled into sachets.

TABLE M

| Strength | 1 mg | 5 mg | 10 mg | 50 mg | 100 mg | 200 mg | 300 mg |
|---|---|---|---|---|---|---|---|
| Formula I (mg) | 1.00 | 5.00 | 10.00 | 50.00 | 100.00 | 200.00 | 300.00 |
| Copovidone (mg) | 6.25 | 6.25 | 6.25 | 12.50 | 25.0 | 17.00 | 25.00 |
| Dibasic calcium | 25.00 | 25.00 | 25.00 | 50.00 | 100.00 | 20.00 | 30.00 |
| phosphate dihydrate | | | | | | | |
| (mg) | | | | | | | |
| Microcrystalline | 283.25 | 280.39 | 277.53 | 251.93 | 212.87 | 161.74 | 98.61 |
| cellulose (mg) | | | | | | | |
| Lactose (mg) | 258.00 | 256.86 | 254.72 | 201.57 | 128.13 | 167.26 | 112.39 |
| Sodium starch glycolate | 17.50 | 17.50 | 17.50 | 25.00 | 225.00 | 25.00 | 25.00 |
| (mg) | | | | | | | |
| Magnesium stearate (mg) | 9.00 | 9.00 | 9.00 | 9.00 | 9.00 | 9.00 | 9.00 |
| Total (mg) | 600.00 | 600.00 | 600.00 | 600.00 | 600.00 | 600.00 | 600.00 |

A further example compression formulation is provided in Table N.

11-Piperazin-1-yldibenzo[b,f][1,4]thiazepine and crospovidone are blended. To this mixture, the silicified microcrystalline cellulose is added and blended further. Next, the sodium stearyl fumarate is added and blended. This mixture is compressed into tablets using a tablet compression machine or filled into capsules using an encapsulation device or filled into sachets.

TABLE N

| Strength | 1 mg | 5 mg | 10 mg | 50 mg | 100 mg | 200 mg | 300 mg | 400 mg |
|---|---|---|---|---|---|---|---|---|
| Formula I (mg) | 1.00 | 5.00 | 10.00 | 50.00 | 100.00 | 200.00 | 300.00 | 400.00 |
| Silicified micro- | 86.00 | 82.00 | 77.00 | 80.50 | 161.00 | 322.00 | 483.00 | 644.00 |
| crystalline | | | | | | | | |
| cellulose (mg) | | | | | | | | |
| Crospovidone | 8.00 | 8.00 | 8.00 | 12.00 | 24.00 | 48.00 | 72.00 | 96.00 |
| (mg) | | | | | | | | |
| Sodium stearyl | 5.00 | 5.00 | 5.00 | 7.50 | 15.00 | 30.00 | 45.00 | 60.00 |
| fumarate (mg) | | | | | | | | |
| Total (mg) | 100.00 | 100.00 | 100.00 | 150.00 | 300.00 | 600.00 | 900.00 | 1200.00 |

### Example 7

### Roller Compression Formulations

11-Piperazin-1-yldibenzo[b,f][1,4]thiazepine is blended with crospovidone. A portion of the microcrystalline cellulose, lactose, and magnesium stearate is added and blended further The blend is compacted using a roller compactor and milled. The remainder of the microcrystalline cellulose and lactose are added and blended. Magnesium stearate is added and blended. This mixture is compressed into tablets using a tablet compression machine or filled into capsules using an encapsulation device or filled into sachets. An example roller compression formulation is provided in Table O.

TABLE O

| Strength | 1 mg | 5 mg | 10 mg | 50 mg | 100 mg | 200 mg | 300 mg | 400 mg |
|---|---|---|---|---|---|---|---|---|
| Formula I (mg) | 1.00 | 5.00 | 10.00 | 50.00 | 100.00 | 200.00 | 300.00 | 400.00 |
| Microcrystalline | 44.75 | 42.75 | 40.25 | 44.88 | 89.75 | 179.50 | 210.75 | 359.00 |
| cellulose (mg) | | | | | | | | |
| Lactose mg) | 44.75 | 42.75 | 40.25 | 44.88 | 89.75 | 179.50 | 210.75 | 359.00 |
| Crospovidone (mg) | 8.00 | 8.00 | 8.00 | 8.00 | 16.00 | 32.00 | 48.00 | 64.00 |
| Magnesium | 1.50 | 1.50 | 1.50 | 2.25 | 4.50 | 9.00 | 13.50 | 18.00 |
| stearate (mg) | | | | | | | | |
| Total (mg) | 100.00 | 100.00 | 100.00 | 150.00 | 300.00 | 600.00 | 900.00 | 1200.00 |

### Example 8

### Powder Formulation for Suspension

The compound of Formula I can be formulated as a powder dosage form that can be converted to a suspension just prior to use or alternatively added to food. An example formulation is provided below in Table U. The formulation is considered to be a free flowing powder with a light bulk density, This formulation can be prepared by using a combination of appropriate excipients such as a binder, a filler, a sweetener, and a suspending agent.

TABLE U

| Ingredient | Amount (wt %) |
|---|---|
| Formula I | 5% |
| Binder | 10% |
| Filler | 75% |
| Sweetener | 5% |
| Suspending Agent | 5% |

### Example 9

### Sustained Release Powder Formulation

Table V below provides an example sustained release formulation. This formula can be made by formulating the above dosage form and then applying a sustained release barrier or a film coat. Alternatively, the formulation can be prepared by first applying a sustained release coating on the drug substance.

TABLE V

| Ingredient | Amount (wt %) |
|---|---|
| Formula I | 5% |
| Binder | 10% |
| Filler | 65% |
| Sweetener | 5% |
| Suspending Agent | 5% |
| Coaling Agent | 10% |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A solid formulation comprising solid 11-piperazin-1-yldibenzo[b,f][1,4]thiazepine and at least one pharmaceutical excipient selected from a filler, a binder, a disintegrant, a suspending agent, a coating agent, a sweetener, a flavoring, and a lubricant.

2. The solid formulation of claim 1 wherein said excipient selected from an inorganic salt filler, a cellulose filler, an oligosaccharide filler, a non-cellulosic binder, a disintegrant, and a lubricant.

3. The solid formulation of claim 1 comprising about 0.1 to about 99% by weight of said 11-piperazin-1-yldibenzo[b,f][1,4]thiazepine.

4. The solid formulation of claim 1 comprising about 0.1 to about 75% by weight of said 11-piperazin-1-yldibenzo[b,f][1,4]thiazepine.

5. The solid formulation of claim 1 comprising about 0.1 to about 50% by weight of said 11-piperazin-1-yldibenzo[b,f][1,4]thiazepine.

6. The solid formulation of claim 1 comprising about 1 to about 25% by weight of inorganic salt filler.

7. The solid formulation of claim 6 wherein said inorganic salt comprises a phosphate salt.

8. The solid formulation of claim 7 wherein said inorganic salt is dibasic calcium phosphate dihydrate.

9. The solid formulation of claim 1 comprising about 10 to about 95% by weight of a cellulose filler.

10. The solid formulation of claim 9 wherein said cellulose filler is microcrystalline cellulose or silicified microcrystalline cellulose.

11. The solid formulation of claim 1 comprising about 15 to about 50% by weight of an oligosaccharide filler.

12. The solid formulation of claim 11 wherein said oligosaccharide filler is lactose.

13. The solid formulation of claim 1 comprising about 0.5 to about 15% by weight of a non-cellulosic binder.

14. The solid formulation of claim 1 wherein said non-cellulosic binder is povidone or copovidone.

15. The solid formulation of claim 1 comprising about 1 to about 15% by weight of a disintegrant.

16. The solid formulation of claim 15 wherein said disintegrant is sodium starch glycolate or crospovidone.

17. The solid formulation of claim 1 comprising about 0.1 to about 8% by weight of a lubricant.

18. The solid formulation of claim 17 wherein said lubricant is magnesium stearate or sodium strearyl fumarate.

19. The solid formulation of claim 1 comprising about 0.1 to about 75% by weight of said 11-piperazin-1-yldibenzo[b,f][1,4]thiazepine; about 1 to about 25% by weight of an inorganic salt filler; about 10 to about 95% by weight of a cellulose filler; about 15 to about 50% by weight of an oligosaccharide filler; about 0.5 to about 15% by weight of a non-cellulosic binder; about 1 to about 15% by weight of a disintegrant; and about 0.1 to about 8% by weight of a lubricant.

20. The solid formulation of claim 19 comprising about 0.1 to about 75% by weight of said 11-piperazin-1-yldibenzo[b,f][1,4]thiazepine; about 1 to about 25% by weight of dibasic calcium phosphate dihydrate; about 10 to about 95% by weight of microcrystalline cellulose or silicified microcrystalline cellulose; about 15 to about 50% by weight of lactose; about 0.5 to about 15% by weight of povidone or copovidone; about 1 to about 15% by weight of sodium starch glycolate or crospovidone; and about 0.1 to about 8% by weight of magnesium stearate or sodium stearyl fumarate.

21. The solid formulation of claim 1 comprising about 0.1 to about 75% by weight of said 11-piperazin-1-yldibenzo[b,f][1,4]thiazepine; about 40 to about 90% by weight of a cellulose filler; about 1 to about 15% by weight of a disintegrant; and about 0.1 to about 8% by weight of a lubricant.

22. The solid formulation of claim 21 comprising about 0.1 to about 75% by weight of said 11-piperazin-1-yldibenzo[b,f][1,4]thiazepine; about 40 to about 90% by weight of silicified microcrystalline cellulose; about 1 to about 15% by weight of crospovidone; and about 0.1 to about 8% by weight of sodium stearyl fumarate.

23. The solid formulation of claim 1 comprising about 0.1 to about 75% by weight of said 11-piperazin-1-yldibenzo[b,f][1,4]thiazepine; about 25 to about 55% by weight of a cellulose filler; about 15 to about 50% by weight of an oligosaccharide filler; about 1 to about 15% by weight of a disintegrant; and about 0.1 to about 8% by weight of a lubricant.

24. The solid formulation of claim 23 comprising about 0.1 to about 75% by weight of said 11-piperazin-1-yldibenzo[b,f][1,4]thiazepine; about 25 to about 55% by weight of microcrystalline cellulose; about 15 to about 50% by weight of lactose; about 1 to about 15% by weight of crospovidone; and about 0.1 to about 8% by weight of a magnesium stearate.

25. The solid formulation according to claim 1 prepared by a wet granulation method.

26. The solid formulation according to claim 1 wherein said 11-piperazin-1-yldibenzo[b,f][1,4]thiazepine is crystalline.

27. An oral dosage form comprising the solid formulation according to claim 1.

28. The oral dosage form of claim 27 which is a tablet, caplet, capsule, or sachet.

29. The oral dosage form of claim 27 comprising about 0.1 mg to about 750 mg of solid 11-piperazin-1-yldibenzo[b,f][1,4]thiazepine.

30. The oral dosage form of claim 27 having a total weight of about 50 to about 1500 mg.

31. A powder suitable for suspension in a liquid vehicle comprising a formulation according to claim 1.
